(19) United States
(12) Patent Application Publication
(10) Pub. No.: US 2023/0303817 A1
(43) Pub. Date: Sep. 28, 2023

(54) BLENDED POLYVINYL ALCOHOL DRUG DELIVERY SYSTEMS

(71) Applicant: The University of British Columbia, Vancouver (CA)

(72) Inventors: John K. Jackson, West Vancouver, British Columbia (CA); David Plackett, Vancouver, British Columbia (CA); Qianyu Chen, Vancouver, British Columbia (CA); Malihesadat Poormasjedimeibod, Vancouver, British Columbia (CA); Helen M. Burt, Vancouver, British Columbia (CA)

(21) Appl. No.: 18/015,157

(22) PCT Filed: Jul. 20, 2021
(86) PCT No.: PCT/CA2021/051004

§ 371 Date: Jan. 9, 2023

Related U.S. Application Data
(60) Provisional application No. 63/055,189, filed on Jul. 22, 2020.

(51) Int. Cl. C08L 29/04; A61K 33/38; A61K 9/00; A61F 13/00

(52) U.S. Cl. CPC C08L 29/04; A61F 13/00017; A61K 9/0014; A61K 33/38; C08L 2205/025

(57) ABSTRACT

The invention provides biocompatible polyvinyl alcohol (PVA) matrices, composed of blends of differently hydrolyzed PVAs. The PVA matrices may be formed into controlled release formulations, for example for topical drug delivery.

### FIELD

The invention relates to the field of local drug delivery over extended periods. More specifically, the invention relates to compositions for the delivery of drug to skin, in particular as a treatment for wounds and skin diseases. Further, the invention relates to methods of preparing the compositions for use in wound healing and topical drug delivery.

### BACKGROUND

Topical administration and controlled release of pharmacologically active substances is important in optimal wound healing and the treatment of skin diseases or conditions such as psoriasis, atopic dermatitis, rosacea or eczema. Sometimes it is necessary to deliver drugs in a controlled fashion and such applications might be best served by a polymeric formulation of one or more drugs whereby the polymer forms a durable hydrogel that releases the drug over a defined period of time but then degrades and is resorbed. Each disease might have a different time frame requirement for such degradation and drug release. Therefore, a suitable platform might be one whereby the degradation rate of the polymer might be tuned to suit that need. The most relevant application for the technology is the delivery of drugs to the skin, especially wound sites, but the technology may be utilized in numerous other disease sites in the body using a wide range of drugs. The goal of a wound dressing is to replicate the function of our skin and promote its regeneration. Our skin protects us, regulates temperature through fluid exchange and helps enable sensation. A wound dressing should be antimicrobial, biocompatible, non-adhesive and pain-free [1].

### Polyvinyl Alcohol (PVA)

PVA is a water-soluble, synthetic polymer that is biocompatible with high tensile strength and flexibility. PVA swells in water to form a hydrogel membrane which creates a moist environment with good gas exchange properties that promotes optimal wound healing [2]. PVA is prepared by hydrolyzing polyvinyl acetate in alcohol in the presence of a base:

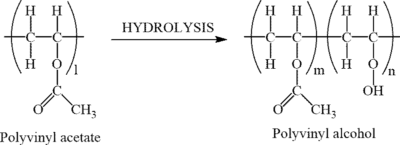

PVA is commercially available in a limited choice of degrees of hydrolysis, reflecting the extent to which ethylene units have been removed and replaced by hydroxyl substituents. The most commonly available forms are 99% hydrolyzed (water insoluble) and 88% hydrolyzed (partially soluble in water). Partially hydrolyzed PVA contains both PVA and unreacted polyvinyl acetate or acetyl groups. Interestingly, the solubility of PVA is inversely proportional to the degree of hydrolyzation whereby cast films made from 99% hydrolyzed PVA swell but dissolve poorly in water and 80% to 90% hydrolyzed PVA films swell but then dissolve rapidly [3]. There are numerous reports of the use of PVA as water solvent cast thin films or as electrospun membranes for application to the skin.

### PVA and Crosslinking of Cast Films

Transparent films may be made by dissolving PVA (e.g., at 10% w/w) in boiling water and then pouring the resulting, cooled viscous solution into a petri dish. However, these films dissolve or disintegrate quickly in water so they could only provide a short residence time on a wound. To overcome this difficulty, various methods have been described to crosslink the PVA to increase the durability of the film, to provide cross-linked PVA films that are non-degradable in water. Crosslinking may involve the use of chemicals such as citric acid [4] or glutaraldehyde [5, 6] but for wound dressing applications these methods need to ensure that all unused chemicals are removed before use. Other methods include e-beam [7] or gamma [8] irradiation which also serve to sterilize the films and freeze thawing [8, 9] which simply allows crystallites to form in the PVA which anchor polymer chains. Other methods for crosslinking that have been investigated include heat, borate, methanol, UV radiation or freeze-thawing. Galeska and colleagues used freeze thawing to effectively crosslink PVA so that films lasted more than 2 weeks in water to release dexamethasone and showed that the rate of drug release was inversely proportional to the degree of crosslinking induced by repeated freeze thawing [10]. To further limit the release of dexamethasone, the authors encapsulated the drug in PLGA microspheres embedded in the freeze-thaw crosslinked films which established this form of crosslinked PVA as a long lasting carrier for extended time periods. Rahmani-Neishaboor and colleagues employed a similar strategy for the release of the protein stratifin [11]. Glutaraldehyde mediated cross linking has been used to produce PVA cast films from PVA with different degrees of hydrolysis [12]. Other workers have blended PVA with other agents to try and produce membranes with improved performance characteristics. The agents include alginate [13, 14], polyvinyl pyrrolidone [3], cellulose derivatives [4, 13, 15] collagen [16], polyethylene glycol [9, 17], chitosan [18] and latex [19]. Jodar et al [2] created blends of PEG 4000 with PVA (98% hydrolyzed) and PVA (88% hydrolyzed) followed by glutaraldehyde crosslinking. These PEG blended films degraded over 1 to 24 hour periods depending on composition allowing for some control of degradation but almost all the silver sulphadiazine anti- infective agent was released within 100 minutes from these films.

Heat crosslinking of PVA has been described extensively in the literature. A disadvantage of using chemical or heat based crosslinking systems for drug delivery is that the drug may be chemically reacted with such agents or heat may degrade the drugs.

### Blended PVA Systems

Numerous workers have used blending methods with PVA to cast films. PVA has been blended with chitosan [20], starch [21], 50:50 blended with Polyvinyl pyrrolidone [22], gelatin [23], cellulose [24], and alginate [25]. For example, Limpan and colleagues blended PVA with fish protein (1:1 ratio) using PVA with different degrees of hydrolysis or different PVA molecular weights and reported on the physical properties of the blended films [26].

### Electrospinning and Wound Dressings

Whilst PVA may be simply solvent cast and dried to form thin films, it has been used extensively in electrospinning non-woven membranes that are well suited for wound dressings [27]. These membranes are very flexible yet strong allowing excellent control of placement and handling. One biological advantage of these nanofibers is that they are of the same scale as biological molecules and are therefore capable of complex interactions with cells [28]. In fact, the structure of nanofibers closely resembles that of extracellular matrix (ECM) [29]. The porous nature of the non-woven nanofiber membrane allows for drainage of wound exudate while still allowing gas exchange [30]. Non-woven nanofiber membranes are an excellent potential drug delivery system. When electrospinning PVA one problem that occurs is that the high surface area to volume ratio allows rapid and extensive hydration in water with 1000% increases in weight followed by rapid dissolution. Therefore, an advantageous electrospun PVA membrane might be rendered slowly degradable to prevent such rapid dissolution. Generally drug delivery with PVA nanofibers has been challenging due to an initial burst release profile leading to a perceived need to crosslink the polymer to inhibit water uptake and drug release [31].

### PVA and Electrospinning

As described above, PVA may be electrospun to form flexible membranes. Generally, the solubility and degradation properties of these membranes should be similar to cast films (although the electrospun membranes with a higher surface area to volume ratio may dissolve more quickly) but the mechanical properties should be quite different to cast films (non-woven membrane vs monolithic cast film). Since the thickness and physical properties of the individual fibers and the density of fibers may affect the mechanical performance of these membranes, numerous workers have studied blending PVA with other agents to affect these properties. Collagen and chitosan-blended PVA electrospun have been characterized for their cell adhesion properties [32, 33]. Similarly Chen and colleagues, combined alginate and PVA in an electrospun membrane [34]. Three groups have electrospun PVA blends using different molecular weight polymers to modify fibre morphology and mechanical properties [35, 36]. Others have compared electrospun membrane made from either 99% hydrolyzed PVA or 88% hydrolyzed PVA individually [37]. Park J-C and colleagues reported various blends of PVA with Poly acrylic acid (PAA) followed by heat treatment to decrease the water solubility of electrospun membrane [38]. In a similar vein, Jannesari M et al electrospun 50:50 ratio blends of PVA with poly vinyl acetate (PVAc) where the PVAc was selected for blending with 98% hydrolyzed PVA [39]. These membranes were used to deliver an antibiotic (ciprofloxacin) to treat wounds where an insoluble membrane that slowly swelled in the aqueous exudate was designed.

### Role of Silver as an Anti-infective in PVA Wound Dressings

Silver is an agent of particular interest in the field of wound healing as it has been shown to be an effective antimicrobial agent and to interact with PVA. Moreover, the combination of silver nitrate and heat has been shown to create silver nanoparticles in the PVA film which is a form of silver preferred by clinicians. PVA is sometimes rendered insoluble by high heat treatment and the added role of silver in such PVA crosslinking is unclear. There are a number of reports describing the use of heat with silver nitrate in PVA to produce silver nanoparticles or nanocables in situ [40-42] in non-degradable films. Luo and colleagues used heat to cross link PVA nanocables and showed that the inclusion of a small amount of silver in the formulation stabilized the nanocables [43].

Jaeghere and colleagues used low % hydrolyzed PVA that was heat extruded as a drug release platform that was almost fully dissolved in 2 hours [44]. Morita and colleagues showed that the inclusion of salts 96-98% hydrolyzed PVA could reduce the % swelling and slow drug release from the PVA [45]. Cozzolini showed that 99% PVA swelled and released drug slower than 88% PVA in water [46].

### SUMMARY

Blended PVA compositions are provided that have been manufactured with PVAs having different degrees of hydrolyzation (for example ranging from 80 to 99%) in various ratios, which allowed for controllable degradation over many days. No heating was required and the inclusion of (i) alternative salts of silver drugs allowed for an effective antimicrobial composition; or (ii) other therapeutic agents allowed for a controlled drug release.

In one aspect of the invention, there is provided a skin or wound care dressing composition composed of blended PVA polymers that feature different degrees of hydrolysis.

In another aspect of the invention, the skin or wound care dressing composition further comprises one or more additional drugs or therapeutic agents.

In other aspects of the invention, the drug may be selected from one or more of an antimicrobial agent, anesthetic agent, an anti-inflammatory agent, an antiproliferative or a wound modulating agent.

In some aspects of the invention, the antimicrobial agent is a silver salt selected from silver nitrate, silver carbonate, silver sulphate, silver acetate or silver sulphadiazine.

In one aspect of the invention, the blended PVA polymers are manufactured from a mixture of partially hydrolyzed PVA (less than 90% hydrolyzed) and fully hydrolyzed PVA (99% hydrolyzed).

In another aspect of the invention, the blended PVA polymers are manufactured from a mixture of partially hydrolyzed PVA (less than 90% hydrolyzed) with intermediate hydrolyzed (90-97% hydrolyzed) PVA.

In another aspect of the invention, the blended PVA polymers are manufactured from a mixture of intermediate hydrolyzed PVA (90-97%) and fully hydrolyzed PVA (99% hydrolyzed).

In some aspects of the invention, the blended PVA polymers provide a slow degradation profile that degrades over 5 days or 10 days.

In another aspect of the invention, the blended PVA polymers provide a continual release of one or more drugs from the polymer over a 5 day, 10 day period or 15 day period.

In another aspect of the invention there is provided a method of manufacturing for the blended PVA polymer based skin or wound care dressing.

In some aspects of the invention, the skin or wound care dressing is made by casting the blended PVA polymers as a film containing one or more therapeutic agents.

In another aspect of the invention the blended PVA polymers may be used for the transdermal delivery of drugs.

In some aspects of the invention, the skin or wound care dressing is made using an electrospinning process to combine the PVA polymers and the one or more therapeutic agents to form a membrane

Methods are accordingly provide for forming a polymer matrix, involving admixing a first polyvinyl alcohol (PVA) polymer with a second PVA polymer, where the first PVA polymer is hydrolyzed to a first degree of hydrolysis of from 80% to 100% and the second PVA polymer is hydrolyzed to a second degree of hydrolysis of from 75% to 96%, and the first degree of hydrolysis is at least 4% higher than the second degree of hydrolysis. The first and second PVA polymers may be present respectively in a blended PVA weight ratio of from 5:95 to 95:5. The methods may also include allowing the admixed PVA polymers to form the polymer matrix, such as a biocompatible hydrogel-forming polymer matrix, where the polymer matrix is at least partially water soluble, and the blended PVA weight ratio is selected to provide a desired degree of water solubility of the matrix. Corresponding polymer matrices are accordingly provided, together with methods of using the polymer matrices, for example to deliver medicaments.

The polymer matrices and methods for making them may for example include one or more of the following features. The method or matrix where the polymer matrix is a hydrogel-forming polymer matrix, forming a hydrogel when appropriately hydrated (the polymer matrix may for example be capable of forming a hydrogel under selected hydration conditions, but may nevertheless be used under conditions in which a hydrogel does not in fact form, matrices of this kind are nevertheless hydrogel-forming matrices in the sense of being capable of forming hydrogels). The admixing may for example be in the substantial absence of a cross linking agent. The first and second PVA polymers may be substantially free of covalent crosslinks therebetween. The admixing may alternatively be by electrospinning, or by casting and drying. The polymer matrix may be substantially free of polyethylene glycol (PEG). Polymers in the polymer matrix may be made up essentially of the first and second PVA polymers, i.e. the matrix may substantially lack additional or alternative polymers (although compounds other than alternative polymers may be present in these embodiments). The first PVA polymer and/or the second PVA polymer may for example have a molecular weight of between 9,000 and 150,000. The first degree of hydrolysis may be from 90% to 99%, 94% to 99%, or about 99%. The second degree of hydrolysis may be less than 99%, 80% to 96%, 88% to 96%, or about 88%. The first degree of hydrolysis may be at least 97% and the second degree of hydrolysis from 90% to 97%; or, the first degree of hydrolysis is 99% and the second degree of hydrolysis is less than 90%; or, the first degree of hydrolysis is 90-97% and the second degree of hydrolysis is less than 90%; or, the first degree of hydrolysis is 99% and the second degree of hydrolysis is 90-97%. Alternatively, the first degree of hydrolysis may be from 90% to 99% and the second degree of hydrolysis below 90%. The first and second PVA polymers may be present respectively in the blended PVA weight ratio of from 10:90 to 50:50. The polymer matrix may optionally further comprises one or more additional distinct PVA polymers, for example where the additional distinct PVA polymers have a degree of hydrolysis that is different from the first and second degrees of hydrolysis.

The polymer matrix may be biocompatible, and may further include a therapeutic agent, a cosmetic agent or a biologically active agent (any agent having a biological acitivity). The therapeutic agent may for example be one or more of an antimicrobial agent, an anesthetic agent, an anti-inflammatory agent, an antiproliferative agent or a wound modulating agent. The therapeutic agent may be a silver salt, such as silver nitrate, silver carbonate, silver sulphate, silver acetate or silver sulphadiazine. The polymer matrix may be a controlled release matrix, for example for the therapeutic agent, the cosmetic agent or the biologically active agent. The controlled release matrix may be adapted so that when applied to a subject, it releases the therapeutic agent, cosmetic agent or biologically active agent over a slow release period, for example of at least 5, 10 or 15 days. The controlled release matrix may be topically applied to the subject, for example when formed into a skin coating or wound dressing. The polymer matrix is accordingly adaptable for use for controlled release of a medicament, including controlled topical release. Similarly, methods are provided for treating a subject for a disease or disorder by applying to the subject the polymer matrix, for example topically, for example where the polymer matrix includes a medicament. Subjects for treatment may be human or veterinary patients, for example where their disease or disorder is a wound or skin lesion.

### BRIEF DESCRIPTION OF FIGURES

FIG. 1. Degradation profile of blended PVA cast films (99% : 88% hydrolyzed) ranging from ratios of 32:68 (99%:88% hydrolyzed) to 46:54 (99%:88% hydrolyzed) containing 1% w/w Silver Sulphadiazine shown as w/w% content of PVA 88% hydrolyzed in the film.

FIG. 2. Degradation profile of blended PVA cast films (99% : 88% hydrolyzed) containing 1% w/w Silver Carbonate shown as w/w% content of PVA 88% hydrolyzed in the film.

FIG. 3. Degradation profile of blended PVA cast films (99% : 88% hydrolyzed) containing 1% w/w Silver Sulphate shown as w/w% content of PVA 88% hydrolyzed in the film.

FIG. 4. Degradation profile of blended PVA cast films (99% : 88% hydrolyzed) containing 1% w/w Silver Acetate shown as w/w% content of PVA 88% hydrolyzed in the film.

FIG. 5. Release of Silver from PVA cast films made from 40:60 (weight ratio) of PVA99%: PVA88% hydrolyzed loaded with 1% w/w of various silver salts.

FIG. 6. Degradation profile of blended electrospun PVA membranes (99% : 88% hydrolyzed) containing 1% w/w Silver Sulphadiazine shown as w/w% content of PVA 88% hydrolyzed in the membrane.

FIG. 7. Degradation profile of blended electrospun PVA membranes (99% : 88% hydrolyzed) containing 1% w/w Silver Carbonate shown as w/w% content of PVA 88% hydrolyzed in the membrane.

FIG. 8. Degradation profile of blended electrospun PVA membranes (99% : 88% hydrolyzed) containing 1% w/w Silver Sulphate shown as w/w% content of PVA 88% hydrolyzed in the membrane.

FIG. 9. Degradation profile of blended electrospun PVA membranes (99% : 88% hydrolyzed) containing 1% w/w Silver Acetate shown as w/w% content of PVA 88% hydrolyzed in the membrane.

FIG. 10. Release of Silver from PVA electrospun membranes made from 10:90 (weight ratio) of PVA99%: PVA88% hydrolyzed loaded with 1% w/w of various silver salts.

FIG. 11. Anti-bacterial (MRSA) activity of silver loaded PVA films made from 40:60 (weight ratio) of PVA99%: PVA88% hydrolyzed loaded with 1% w/w of various silver salts, or, electropsun membranes made from 10:90 (weight ratio) of PVA99%: PVA88% hydrolyzed loaded with 1% w/w of various silver salts. All four films and four membranes (for all four salts: silver sulphadiazine, carbonate, sulphate and acetate) killed all MRSA bacteria whereas the PVA control film (no silver) had no effect on bacterial growth.

FIG. 12. Electrospinning apparatus and membranes. (A) Shows the nanofibre electrospinning unit (Kato) and (B) show a electrospun PVA membrane (10%PVA (99%) : 90% PVA (88%) containing silver carbonate (1%). The inserts show the final 50 µm thick slightly brown membrane on and a high magnification photo showing the nanofibre network.

FIG. 13. Release of docetaxel from electrospun membranes of blended PVA. The blended PVA membranes were manufactured by electrospinning PVA blends containing either

35:65 (diamonds), 45:55 (squares) or 55:45 (triangles) ratios of PVA 99% hydrolyzed: PVA 88% hydrolyzed and 1% docetaxel. The level of drug release was measured over 7 days.

FIG. 14. Release of gentamicin from cast films of blended PVA. The cast films were manufactured with PVA blends containing either 35:65 (diamonds), 45:55 (squares) or 55:45 (triangles) ratios of PVA 99% hydrolyzed: PVA 88% hydrolyzed and 1% gentamicin. The level of drug release was measured over 7 days.

FIG. 15. Release of gentamicin from electrospun membranes of blended PVA. The electrospun membrane were manufactured with PVA blends containing either 35:65 (diamonds), 45:55 (squares) or 55:45 (triangles) ratios of PVA 99% hydrolyzed: PVA 88% hydrolyzed and 1% gentamicin. The level of drug release was measured over 6 days.

FIG. 16: Release of doxycycline from electrospun membranes of blended PVA. The electrospun membrane were manufactured with PVA blends containing either 35:65 (diamonds), 45:55 (squares) or 55:45 (triangles) ratios of PVA 99% hydrolyzed: PVA 88% hydrolyzed and 1% doxycycline. The level of drug release was measured over 10 days.

FIG. 17. Release of BSA protein from blended PVA electrospun membrane. The electrospun membrane were manufactured with PVA blends containing either 35:65 (diamonds), 45:55 (squares) or 55:45 (triangles) ratios of PVA 99% hydrolyzed: PVA 88% hydrolyzed and 1% BSA. The level of BSA release was measured over 6 days.

FIG. 18. Release of BSA protein from blended PVA cast films. The cast films were manufactured with PVA blends containing either 35:65 (triangles), 45:55 (squares) or 55:45 (triangles) ratios of PVA 99% hydrolyzed: PVA 88% hydrolyzed and 1% BSA. The level of BSA release was measured over 6 days.

### DETAILED DESCRIPTION

The ability to control the release of drugs is important for an effective skin or wound care dressing. Furthermore, the ability to control the degradation time of the PVA dressing may offset the need for repeated dressing changes, may reduce patient morbidity and may be a further mechanism to control drug release. Methods are accordingly provided to control the degradation rate of a PVA-based film or membrane, and thereby modulate the release of substances from PVA matrix, for example in the form of cast films or PVA electrospun membranes.

Methods are provided to blend solutions of high (e.g. approx. 99%) and low (e.g. approx. 88%) hydrolyzed PVA and to cast films that, when dry, have various degrees of solubility. In select embodiments, the degradation rate of such films may be finely controlled by adjusting the percentage of a more soluble form of PVA (e.g. 88%) in a less soluble form of PVA (e.g. 99%). These methods do not require the presence of silver or the use of heat for cross-linking. These blended films may for example be used for the controlled release of many drugs including but not limited to silver. Examples herein illustrate the ability to control the disintegration rate by blending 88% and 99% hydrolyzed PVA. Examples herein also demonstrate the controlled release of various drugs, including numerous silver salts along with drugs of decreasing water solubility: protein biologicals, gentamicin (antibiotic), doxycycline (antibiotic) and docetaxel (antiproliferative) are included.

Methods are also provided for blending the differently hydrolyzed PVAs in the manufacture of electrospun membranes. Such membranes exhibit similar properties to cast films except that the amount of highly, e.g. 99%, hydrolyzed PVA incorporation required to mediate slow degradation is much lower than that needed for use in cast dried films. Electrospun membranes exhibit distinct swelling, degradation and drug release profiles from those found for cast films.

In the exemplified embodiments, in both cast films and electrospun membranes compositions containing more than 49% of the 99% hydrolyzed PVA with less than 51% of the 88% hydrolyzed PVA degraded very slowly. In select embodiments, PVA blends are accordingly provided of 99% and 88% hydrolyzed PVAs, containing less than 49% of the 99% hydrolyzed PVA. The degree of hydrolyzation of PVA may be defined for reference herein as follows: below 90% hydrolyzed: “partial”; between 90 and 97% hydrolyzed: “intermediate”; and, above 97% hydrolyzed: “fully hydrolyzed”. Although 99% and 88% are by far the most commonly available commercial forms of PVA other forms are available. In some aspects, blends that combine intermediately hydrolyzed PVAs (e.g. 92%, 94% or 96%) with either Partial or Fully hydrolyzed are shown to also allow for control of degradation rates.

### EXAMPLES

### Methods

Poly(vinyl alcohol) (Selvol™ 540: 88 mole % hydrolyzed, molecular weight 150,000, Selvol™ 125: 99 mole% hydrolyzed, molecular weight 125,000, Selvol™ 425: 96 mole % hydrolyzed, Selvol™ 418: 92 mole % hydrolyzed, molecular weight 50,000 and Selvol™ 443: 94 mole % hydrolyzed, molecular weight 150,000) was obtained from Sekisui Specialty Chemical Company, Dallas TX. USA). Silver salts, docetaxel, doxycycline, bovine serum albumin (BSA), gentamicin and poly(vinyl alcohol) 80% hydrolyzed, molecular weight 8000 were purchased from Sigma-Aldrich (St. Louis, MO, USA). All chemicals were used as supplied and without further purification. Deionized water was used in the preparation of all experimental PVA-silver formulations.

### Film Preparation (Solvent Cast PVA)

PVA was prepared as a 10% w/w stock solution by slowly adding PVA powder to a suitable volume of rapidly stirred water preheated to 85-90° C. followed by continued stirring and heating for approximately 60 minutes. When a clear solution had formed the vessel was removed from heating and cooled to room temperature. Stock silver salt solutions were prepared in water and stored covered with aluminum foil in a dark cupboard until required. Solutions of PVA were diluted down to 5% w/w and mixed together at the appropriate ratios. Finally, a small volume of the concentrated silver salt solution was then added in sufficient quantity to allow films to be cast in 60 × 15 mm disposable polystyrene Petri dishes to a final thickness of 100um (Sarstedt Inc., Montreal, QC, Canada). Generally, the % of silver ion (not the total wt. of the salt) to PVA was 1%. The PVA-silver solutions in Petri dishes were loosely covered with aluminum foil and left in a 37° C. oven overnight in order for water to evaporate. All dried films were stored in a dark cupboard before evaluation.

### Manufacture of Electrospun Membranes

PVA electrospun membranes were manufactured using a Nanofibre Electrospinning Unit from Kato Tech Co. Ltd. Japan using 10 ml of a 10% PVA polymer solution in water (no glycerol) containing silver salts where the ratio of the blend is described by the percentage of the 99% hydrolyzed PVA to the percentage of the 88% hydrolyzed PVA. In some membranes the two PVA polymers were as follows: (% hydrolyzed) 96:88, 94:88 or 99:94. Films were electrospun overnight (30 KV, 15 cm range, 0.1 mm/min syringe flow rate) and collected onto aluminum foil and stored at room temperature in the dark.

### Film Swelling and Degradation Studies

PVA films or electrospun membranes were prepared as described above. These films were then stored for one week in the dark before use. Small sections of films (approximate diameters of 2 cm) were then placed on moist 0.2 µm filter discs (Millipore, Billerica, MA, USA) and weighed. The films and filters were covered with a thin layer of deionized water and left for appropriate times. After set time points the filter discs and adherent PVA-silver gel were moved to a Millipore vacuum apparatus and all excess water was removed from the filter over approximately 15 seconds. The combined PVA gel and filter were reweighed and recovered with a fresh layer of excess water. The weight gain (swelling) and weight loss (dissolution) were then calculated as a percentage of the original dry film weight.

### Silver Release Studies and Characterization

Films or electrospun membranes containing silver were placed in deionized water and the media sampled at regular intervals for silver analysis by Inductively coupled plasma analysis. Silver calibration standards were run every 10 samples. The instrument held reproducible standard curves over 100 sequential silver analysis with detection limits approximating 10 ng/ml. Each release study was run in triplicate for at least two weeks and the results plotted as the calculated percent silver released as a function of time.

### Antimicrobial Efficacy of Silver-loaded PVA Films

In order to illustrate the antimicrobial activity of PVA-silver formulations, all cast films and electrospun membranes were weighed at 20 mg +/- 0.5 mg. The methicillin resistant strain of S. aureus (MRSA strain USA300) was cultured overnight from freezer stocks (20 µL bacterial sample into 20 mL Luria Bertani (LB) broth), followed by a second sub-culture (200 µL in 20 mL LB broth) until an optical density of -0.3-0.5 at λ=600 nm was reached (measured using an Eppendorf BioPhotometer (Eppendorf, Mississauga, ON, Canada), at which point bacteria were used as outlined below.

The antibacterial activity of PVA-silver films was assessed by placing preweighed (20 mg) film or electrospun membrane samples containing 1% w/w silver loadings of either silver acetate, sulphadiazine, carbonate or sulphate in nutrient media containing Methicillin resistant S. aureus (5.00E + 05 CFU/mL) with 10 mL of 100% culture media per bottle. The sample bottles were incubated at 37° C. with shaking at 100 rpm for 48 hours. Aliquots were taken at 0, 6, 24 and 48 hours after inoculation and bacterial numbers were determined as colony-forming units per mL (CFU) at each time point.

### Encapsulation of Other Therapeutic Agents in PVA Cast Films or Electrospun Membranes.

Films or electrospun membrane were manufactured as described above containing either docetaxel, doxycycline, gentamicin or bovine serum albumin (BSA; as a protein model for any “biological” based drug such as an antibody). The blended films contained either 35%, 45% or 55% PVA (99% hydrolyzed) content. For the drug release studies, Docetaxel was analyzed using HPLC (232 nm, C18, 58/37/5 acetonitrile/water/methanol mobile phase, 20 µL injection, 1 mL per minute) and doxycycline was analyzed similarly (absorbance of 360 nm with a mobile phase of 30% acetonitrile with 70% 10 M phosphate buffer pH 2.8). Gentamicin was analyzed using a fluorescence tag assay using Fluoraldehyde™ (Thermo Fisher) and BSA protein release was assayed using a BCA protein analysis kit.

### Example 1 Degradation of Blended PVA Cast Films

### Film Casting

The use of 5% PVA solutions PVA polymers in water containing 2.5% glycerol was found to provide a method of manufacture so that films were uniformly thin (approx. 100 µm), flexible without cracking. Generally, films were a semi-opaque white colour with the silver carbonate films being slightly brown (which was the colour of the silver salt itself). Silver sulphadiazine is insoluble in water so these films had observable small particles embedded in the films but with a homogeneous dispersion still present in the final dried film.

### Degradation of PVA Cast Films.

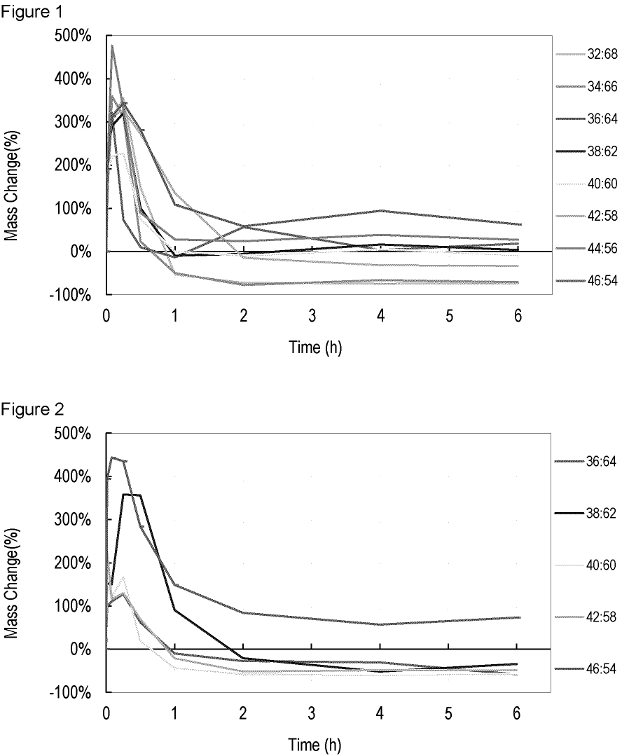

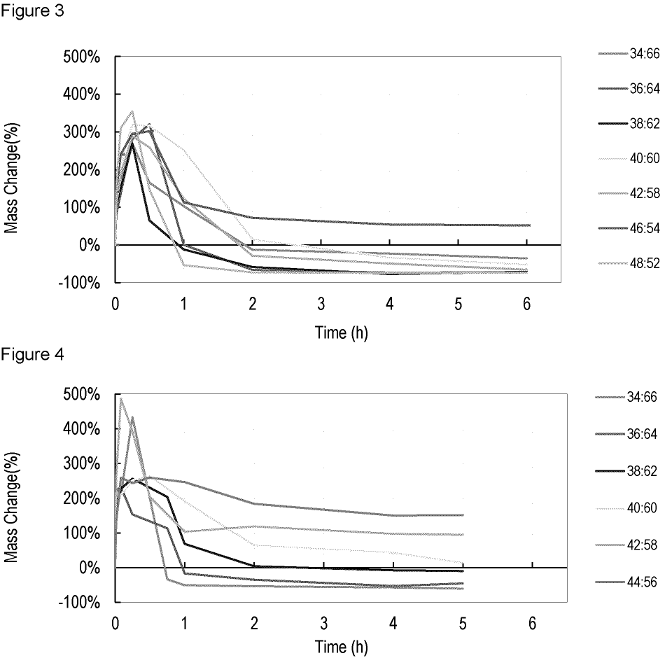

Cast films made from PVA with different degrees of hydrolyzation broke up or dissolved at different rates so that films made from pure 80%, 88%, 92% and even 94% hydrolyzed PVA dissolved in water by 4 hours with 94% being the slowest to dissolve. Films made from 96% hydrolyzed PVA dissolved over more than one day and those with higher degrees of hydrolyzation were essentially insoluble (data not shown). Therefore, a range of PVA blend ratios (99%:88%) of 32:68 to 46:54 was used in these studies. Initially films were cast without silver salts to assess general blend degradation properties. It was observed that films containing 50% or more of the PVA 99% hydrolyzed type did not fully dissolve over a week but those containing just 30% did dissolve. Generally, for all four silver salts, the films swelled rapidly to approximately 400% and declined to less than 150% within 2 hours. Subsequently, swelling levels tended to stabilize and drop only slowly over the next few hours. Data is shown for cast films containing silver sulfadiazine, silver carbonate, silver sulphate and silver acetate in FIGS. 1-4. Furthermore, films containing PVA 99% hydrolyzed contents greater than 40% did not fully degrade and swelling remained above 0%.

Note that a 0% swelling means the weight of the film is the same as the dry weight so some material has been lost as some water is present in the remaining film. To fully dissolve, a value of -100% weight must be attained and none of the films were fully dissolved after 13 days. However, films at -50% weight were no longer intact and very fragmented.

The swelling studies were followed for over ten days with weights being taken at 1,2 6 and 13 days. Most of the values remained the same as at 6 hours so this data is not shown so as not to compact the x axis graph.

There were some noticeable differences in swelling between PVA films containing different silver salts. Films with either the acetate or sulphadiazine salts were more robust and less soluble at 1 hour with lower PVA 99% and higher PVA 88% hydrolyzed content than films made with sulphate or carbonate salts. For example, silver acetate containing films with PVA 99% contents of 44%, 42% and 40% all stayed at or close to 0% swollen at 6 hours (and through to 13 days, data not shown) whereas for the sulphate salt only 48% and 46% PVA 99% content films remained above the 0% swelling point.

When the lower partially hydrolyzed PVA (80% hydrolyzed) was substituted for the 88% hydrolyzed PVA, films containing 30% content of the 99% hydrolyzed PVA only swelled to 200% and then began to degrade after 30 minutes (data not shown) with 150% swelling remaining at 4 hours (Table 1). Films containing 40% or 50% content of the PVA 99% hydrolyzed swelled to 300 and 360%, respectively, and remained undegraded at 4 hours (Table 1).

In other studies where 96% intermediate hydrolyzed PVA was substituted for the fully hydrolyzed PVA (99% hydrolyzed), a blend ratio-dependent control of degradation was observed. Films containing just 30% content of the 96% hydrolyzed PVA began to degrade in one hour with almost all the film dissolved at 4 hours, whereas films containing 40% or 50% content of the 96% hydrolyzed PVA showed considerable swelling and degraded slowly over 9 days (Table 1).

In studies where the fully hydrolyzed PVA (99% hydrolyzed) was blended with intermediate hydrolyzed PVA (either 94% or 92% hydrolyzed,) a blend ratio control of degradation was observed such that at 10% (not shown) or 20% content levels of the 99% hydrolyzed PVA, all films degraded and dissolved quickly whereas at 30 and 40% levels there was slower or little degradation observed over 72 hours (Table 1).

TABLE 1

PVA film degradation (w/w% of original film prior to placing in water)

| % PVA Hydrolyzation | | 20:80 (PVA1:PVA2) | | 30:70 (PVA1:PVA2) | | 40:60 (PVA1:PVA2) | | 50:50 (PVA1:PVA2) | |
|---|---|---|---|---|---|---|---|---|---|
| PVA1 | PVA2 | 4 hours | 3 days | 4 hours | 3 days | 4 hours | 3 days | 4 hours | 9 days |
| 99 | 80 | | | 150 | | 300 | | 360 | |
| 96 | 88 | | | -70 | -70 | 700 | 100 | 810 | 190 |
| 99 | 92 | -100 | -100 | 405 | 240 | 530 | 440 | | |
| 99 | 94 | 50 | -100 | 505 | 80 | 595 | 390 | | |

Overall these data show that control of film degradation may be achieved by adjusting the blending ratios of two different PVA compositions: a fully hydrolyzed with a partially hydrolyzed, an intermediate hydrolyzed with a partially hydrolyzed, and a fully hydrolyzed with an intermediate hydrolyzed. Furthermore, these data demonstrate that fine control of film degradation may be achieved for films containing each of four silver salts by changing the blending % of PVA (99:88% hydrolyzed) where thePVA 99% hydrolyzed content is in the range of 30% to 50% by weight to PVA 88% hydrolyzed.

### Example 2 Degradation of Blended Electrospun PVA Membranes

### Membrane Electrospinning

Using the described electrospinning methods, PVA blends containing 1% silver salts produced strong, tissue-like, thin membranes composed of nanofibres with a diameter of approximately 700 nm. The type of silver salt had little impact on the characteristics of the membranes except that the silver carbonate films were slightly brown (like the cast films). These tissue-like membranes were robust and handleable with a strength lying between plastic wrap used in kitchens and tissue paper. The membranes were not statically self-adhering but were thin and very flexible and it was not advisable to squeeze them into a ball or they were difficult to smooth out again. Because of the inherent flexibility, glycerol was not included in the preparation. Overall, if packaged on a backing paper (or made thicker) these membranes could be easily applied or stretched over a wound. The membranes were very strong so that even ultrathin materials resisted tearing and self-adherence (FIG. 12).

### Degradation of Electrospun PVA Membranes

Initial studies using electrospun membranes without silver revealed that (compared to cast films) much higher levels of PVA 88% could be included in the PVA membranes without swelling/degradation levels dropping to 0% or lower. Therefore, silver salt loaded membranes were electrospun using PVA (99% hydrolyzed) content of 50% to 0% (i.e. with PVA (88% hydrolyzed) of 50% to 100%).

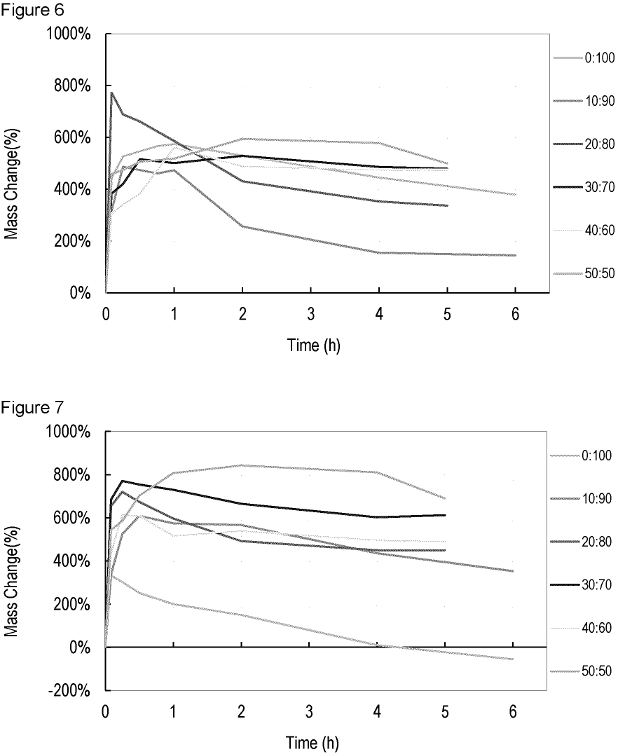

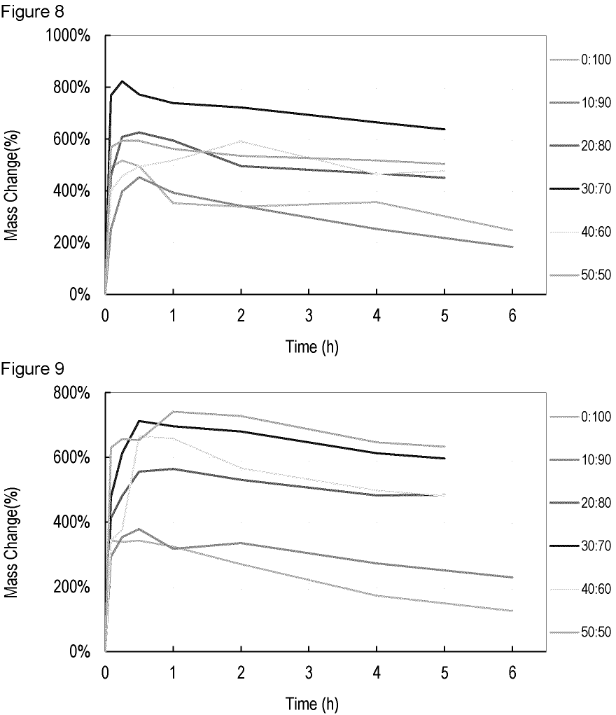

Generally, electrospun membranes composed of blends of PVA (99% hydrolyzed) and PVA (88% hydrolyzed) swelled more than the solvent cast films. All compositions (except those containing 100% w/w PVA(88%)) initially swelled to between 500% and 800% with only minor and slow reductions in swelling over the next 6 hours (FIGS. 6-9). For all salts, membranes containing PVA (99% hydrolyzed) contents of 20% or more remained swollen at levels over 100% for more than 11 days (data points at 1,2,4,7 and 11 days not shown to avoid x axis compaction as data points were largely unchanged beyond 12 hours.

Electrospun membranes spun from blends of 96% and 88% hydrolyzed PVA showed a blend ratio-dependent control of degradation (Table 2). Membranes containing 5% or 10% of the 96% hydrolyzed PVA degraded quickly but membranes with 20% or 30% content of the 96% hydrolyzed PVA degraded from approximately 1100% swollen levels (not shown) to approximately 400% levels at 5 hours (Table 2). Similar results were obtained for membranes spun from 94% intermediate hydrolyzed PVA blended with 88% hydrolyzed where 10% and 20% levels of the 94% hydrolyzed were associated with nearly full degredation at 24 hours, whereas the membranes containing 30% of the 94% PVA were still 200% swollen at 24 hours (Table 2). When 94% hydrolyzed PVA (intermediate) was electrospun with 99% hydrolyzed PVA there was a blend ratio control of degradation observed such that at 30% levels of the 99% hydrolyzed PVA, membranes were non degraded at 24 (Table 2) and 50 hours (not shown) whereas membranes spun using 5 or 10% content levels of the 99% hydrolyzed PVA were significantly degraded by 24 hours (Table 2).

TABLE 2

Electrospun PVA membrane degradation (w/w% of original film prior to placing in water)

| %PVA hydrolyzation | | 5:95 (PVA1:PVA2) | | 10:90 (PVA1:PVA2) | | 20:80 (PVA1:PVA2) | | 30:70 (PVA1:PVA2) | |
|---|---|---|---|---|---|---|---|---|---|
| PVA1 | PVA2 | 5 hours | 24 hours | 5 hours | 24 hours | 5 hours | 24 hours | 5 hours | 24 hours |
| 96 | 88 | 60 | | -70 | | 410 | | 390 | |
| 94 | 88 | | | 60 | -65 | 60 | -60 | 340 | 200 |
| 99 | 94 | 500 | 0 | 200 | 0 | | | 2900 | 2900 |

### Example 3 Release of Silver Salts From Blended PVA Electrospun Membranes

### Silver Release Studies

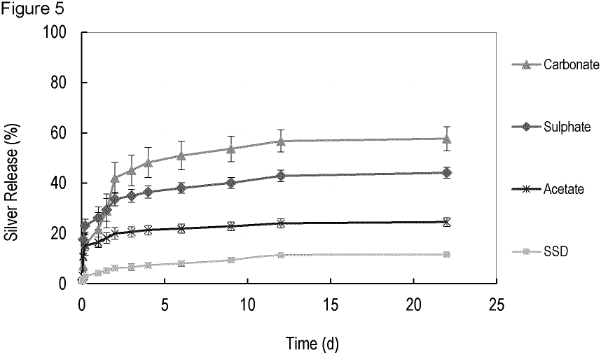

The release of silver salts from solvent cast films is shown in FIG. 5. The order of levels of drug release were carbonate>sulphate>acetate>sulphadiazine. All salts released from electrospun membranes with a burst phase of release in the 15% to 25% range (just 3% for sulphadiazine) followed by a steady sustained rate of release over 3 days followed by a slower but prolonged release of silver over 22 days.

The release of silver salts from electrospun membranes is shown in FIG. 10. The profile of release was similar to that observed for the solvent cast films. There was a burst phase of release of between approximately 15% and 35% followed by a steady phase of release to day 3 (27% to 75%) followed by a slower prolonged release to day 11. As for cast films, the carbonate containing membrane released the most drug with sulphate followed by acetate release rates below that. Opposite to films, electrospun membranes containing silver sulphadiazine released silver very rapidly (with the same kinetics as silver from carbonate membranes). For carbonate and sulphadiazine membranes, almost 100% silver release was achieved by day 11 whereas less silver was released for sulphate (75%) or acetate (45%) at the same time.

### Antibiotic Activity

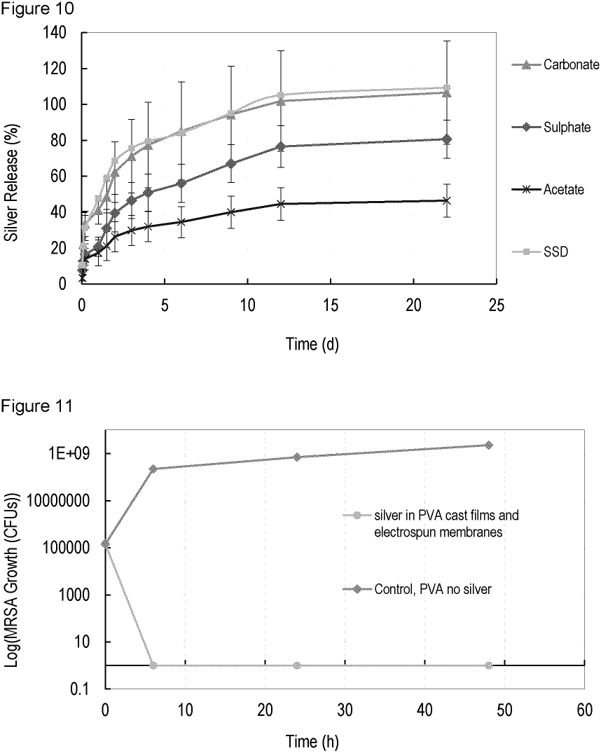

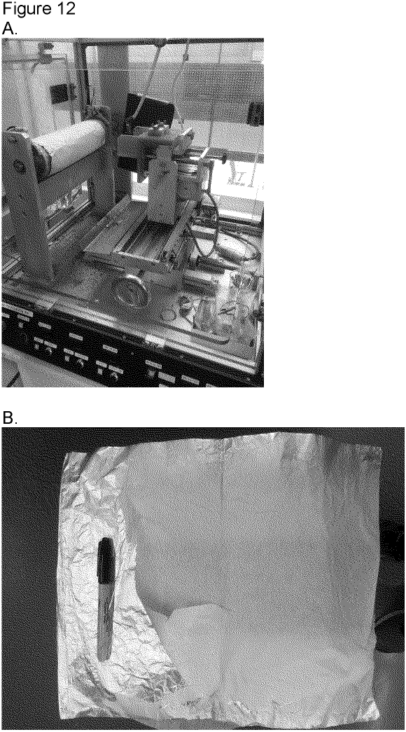

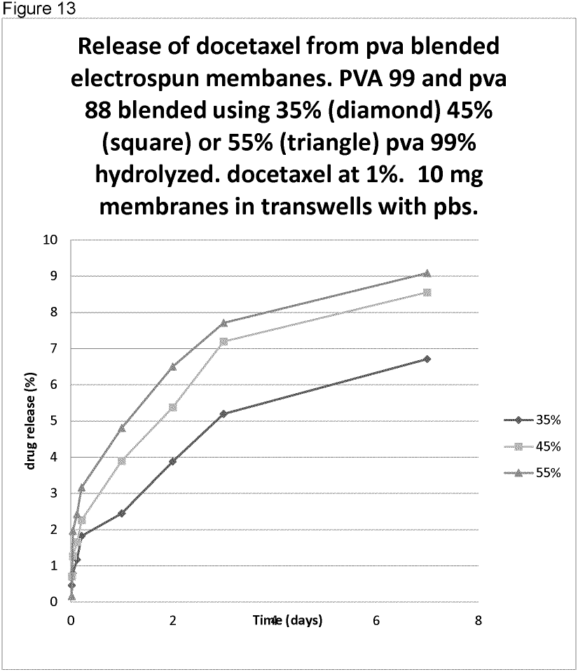

All cast films and electrospun membranes were bactericidal (100% bacterial death) as shown in FIG. 11. The antibacterial activity was common to all silver sulphadiazine, carbonate, sulphate and acetate salts. PVA films containing no silver had no effect on the rapid growth of bacteria.

### Example 4. Release of Other Drugs From Blended PVA Cast Films or Electrospun Membranes.

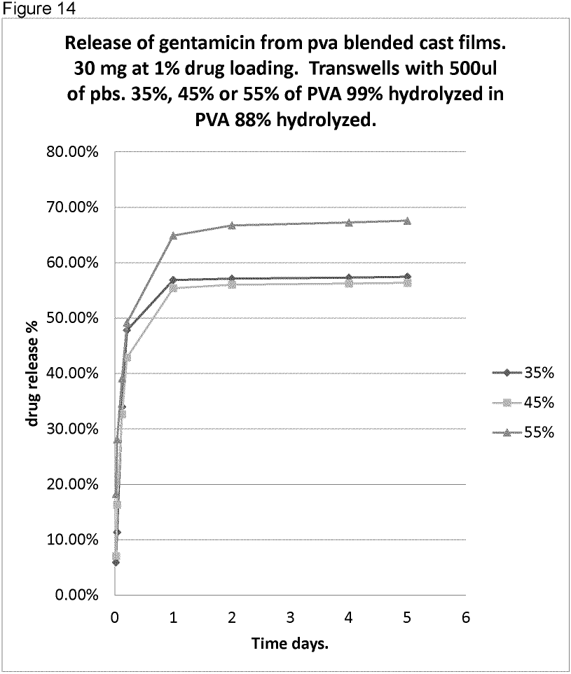

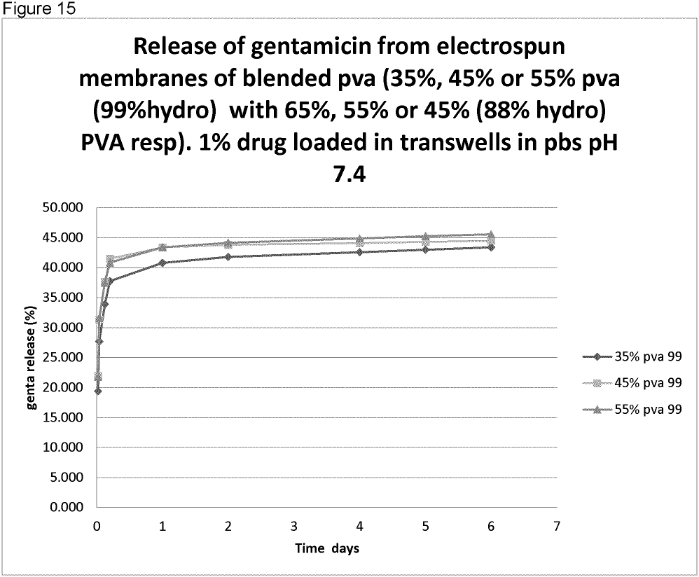

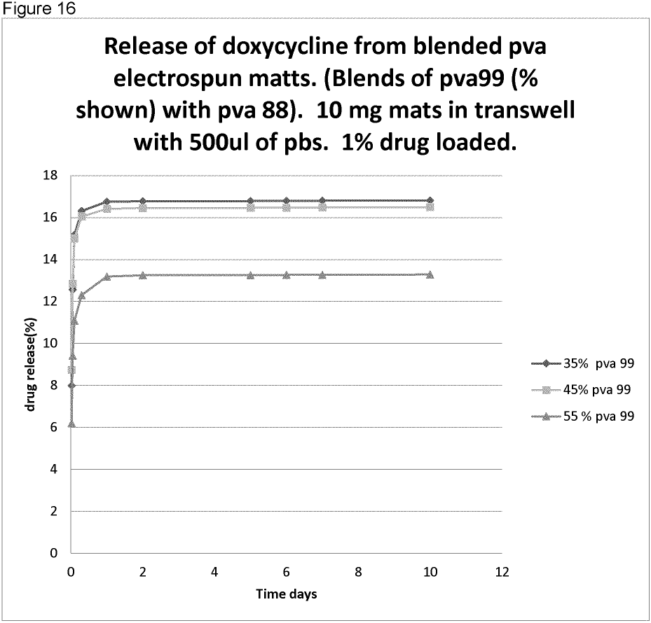

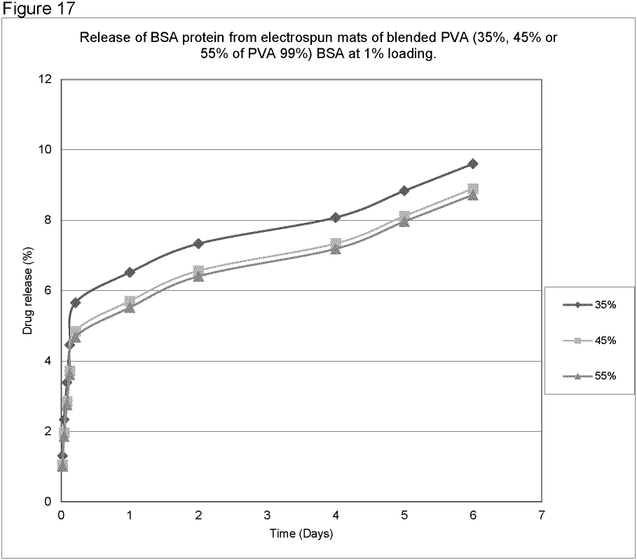

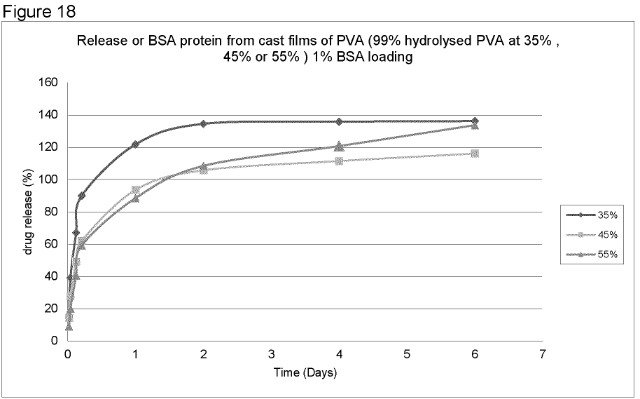

Collectively, the results in FIGS. 13-18 show that both cast and electrospun films made from blends of PVA with 99% and 88% hydrolyzed PVA could provide sustained drug released over a number of days. Docetaxel release reached approximately 5-8% of total drug from the electrospun PVA membranes (FIG. 13). Approximately 50-70% of gentamicin was released from cast PVA films (FIG. 14) and 40% of gentamicin was released from the electrospun PVA films (FIG. 15). Lower levels of doxycycline release were evident from electrospun PVA films ranging from 12-16% (FIG. 16). BSA protein released slowly from electrospun membrane of blended PVA (10% release at 6 days) whereas it released rapidly from cast films (100% release at day 6) (FIGS. 17-18).

In summary, the present examples demonstrate that blended membranes of PVA may be used as controlled release systems for numerous drugs ranging in water solubility from silver nitrate and BSA (Freely soluble) to gentamicin (50 mg/ml), silver acetate (11 mg/ml), silver sulphate (approx. 2 mg/ml), doxycycline (500 ug/ml), silver carbonate 40 ug/ml, silver sulphadiazine (5 ug/ml) and docetaxel (4 ug/ml). Electrospun blended PVA membranes offer the same ability to control degradation profiles as cast films but they have the clear advantage of potentially being a lightweight, easy to apply membrane which have a large capacity to absorb exudate over a long period of time without significant weight loss. Furthermore, the release profiles of silver for all four salts demonstrated near perfect sustained release for nearly two weeks.

### DEFINITIONS AND REFERENCES

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Terms such as “exemplary” or “exemplified” are used herein to mean “serving as an example, instance, or illustration.” Any implementation described herein as “exemplary” or “exemplified” is accordingly not to be construed as necessarily preferred or advantageous over other implementations, all such implementations being independent embodiments. Unless otherwise stated, numeric ranges are inclusive of the numbers defining the range, and numbers are necessarily approximations to the given decimal. The word “comprising” is used herein as an open-ended term, substantially equivalent to the phrase “including, but not limited to”, and the word “comprises” has a corresponding meaning. As used herein, the singular forms “a”, “an” and “the” include plural referents unless the context clearly dictates otherwise. Thus, for example, reference to “a thing” includes more than one such thing.

Citation of references herein is not an admission that such references are prior art to the present invention. Any priority document(s) and all publications, including but not limited to patents and patent applications, cited in this specification, and all documents cited in such documents and publications, are hereby incorporated herein by reference as if each individual publication were specifically and individually indicated to be incorporated by reference herein and as though fully set forth herein. The invention includes all embodiments and variations substantially as hereinbefore described and with reference to the examples and drawings.
1. Selig, H.F., et al., The properties of an “ideal” burn wound dressing--what do we need in daily clinical practice? Results of a worldwide online survey among burn care specialists. Burns, 2012. 38(7): p. 960-6.
2. Sood, A., M.S. Granick, and N.L. Tomaselli, Wound Dressings and Comparative Effectiveness Data. Adv Wound Care (New Rochelle). 2014 Aug 1;3(8):511-529.
3. Teodorescu, M., M. Bercea, and S. Morariu, Biomaterials of PVA and PVP in medical and pharmaceutical applications: Perspectives and challenges. Biotechnology Advances, 2019. 37(1): p. 109-131.
4. Wali, A., et al., Electrospinning of non-ionic cellulose ethers/polyvinyl alcohol nanofibers: Characterization and applications. Carbohydrate Polymers, 2018. 181: p. 175-182.
5. Augustine, R., et al., Electrospun polyvinyl alcohol membranes incorporated with green synthesized silver nanoparticles for wound dressing applications. J Mater Sci Mater Med, 2018. 29(11): p. 163.
6. Jodar, K.S., et al., Development and characterization of a hydrogel containing silver sulfadiazine for antimicrobial topical applications. J Pharm Sci, 2015. 104(7): p. 2241-54.
7. Gao, D., et al., Preparation and Characterization of Silver Sulfadiazine-Loaded Polyvinyl Alcohol Hydrogels as an Antibacterial Wound Dressing. J Pharm Sci, 2018. 107(9): p. 2377-2384.
8. Oliveira, R.N., et al., Mechanical properties and in vitro characterization of polyvinyl alcohol-nano-silver hydrogel wound dressings. Interface Focus, 2014. 4(1): p. 20130049.
9. Ahmed, A.S., et al., PVA-PEG physically cross-linked hydrogel film as a wound dressing: experimental design and optimization. Pharm Dev Technol, 2018. 23(8): p. 751-760.
10. Galeska, I., et al., Controlled release of dexamethasone from PLGA microspheres embedded within polyacid-containing PVA hydrogels. The AAPS Journal, 2005. 7(1): p. E231-E240.
11. Rahmani-Neishaboor, E., et al., Localized controlled release of stratifin reduces implantation-induced dermal fibrosis. Acta Biomater, 2012. 8(10): p. 3660-8.
12. Mansur, H.S., et al., FTIR spectroscopy characterization of poly (vinyl alcohol) hydrogel with different hydrolysis degree and chemically crosslinked with glutaraldehyde. Materials Science and Engineering: C, 2008. 28(4): p. 539-548.
13. Anirudhan, T.S., A.S. Nair, and S.G. S, The role of biopolymer matrix films derived from carboxymethyl cellulose, sodium alginate and polyvinyl alcohol on the sustained transdermal release of diltiazem. Int J Biol Macromol, 2018. 107(Pt A): p. 779-789.
14. Kanimozhi, K., et al., Development and Characterization of Sodium Alginate/Poly(vinyl alcohol) Blend Scaffold with Ciprofloxacin Loaded in Controlled Drug Delivery System. J Nanosci Nanotechnol, 2019. 19(5): p. 2493-2500.
15. Kazsoki, A., et al., Microstructural characterization of papaverine-loaded HPC/PVA gels, films and nanofibers. European Journal of Pharmaceutical Sciences, 2018. 122: p. 9-12.
16.Wang, M., et al., Preparation and characterization of novel poly (vinyl alcohol)/collagen double-network hydrogels. International Journal of Biological Macromolecules, 2018. 118: p. 41-48.
17. Rolim, W.R., et al., Antimicrobial Activity and Cytotoxicity to Tumor Cells of Nitric Oxide Donor and Silver Nanoparticles Containing PVA/PEG Films for Topical Applications. ACS Appl Mater Interfaces, 2019. 11(6): p. 6589-6604.
18. Morgado, P.I., et al., Ibuprofen loaded PVA/chitosan membranes: A highly efficient strategy towards an improved skin wound healing. Carbohydr Polym, 2017. 159: p. 136-145.
19. Ramos, M.V., et al., Wound healing modulation by a latex protein-containing polyvinyl alcohol biomembrane. Naunyn Schmiedebergs Arch Pharmacol, 2016. 389(7): p. 747-56.
20. Costa-Júnior, E.S., et al., Preparation and characterization of chitosan/poly(vinyl alcohol) chemically crosslinked blends for biomedical applications. Carbohydrate Polymers, 2009. 76(3): p. 472-481.
21. Jayasekara, R., et al., Biodegradation by Composting of Surface Modified Starch and PVA Blended Films. Journal of Polymers and the Environment, 2003. 11(2): p. 49-56.
22. Abdelrazek, E.M., I.S. Elashmawi, and S. Labeeb, Chitosan filler effects on the experimental characterization, spectroscopic investigation and thermal studies of PVA/PVP blend films. Physica B: Condensed Matter, 2010. 405(8): p. 2021-2027.
23. Silva, G.G.D., et al., Biodegradable Films Based on Blends of Gelatin and Poly (Vinyl Alcohol): Effect of PVA Type or Concentration on Some Physical Properties of Films. Journal of Polymers and the Environment, 2008. 16(4): p. 276-285.
24. Nishio, Y. and R.S.J. Manley, Cellulose-poly(vinyl alcohol) blends prepared from solutions in N,N-dimethylacetamide-lithium chloride. Macromolecules, 1988. 21(5): p. 1270-1277.
25. Miura, K., et al., Thermal and viscoelastic properties of alginate/poly(vinyl alcohol) blends cross-linked with calcium tetraborate. Carbohydrate Polymers, 1999. 39(2): p. 139-144.
26. Limpan, N., et al., Influences of degree of hydrolysis and molecular weight of poly(vinyl alcohol) (PVA) on properties of fish myofibrillar protein/PVA blend films. Food Hydrocolloids, 2012. 29(1): p. 226-233.
27. Frenot, A. and I.S. Chronakis, Polymer nanofibers assembled by electrospinning. Current Opinion in Colloid & Interface Science, 2003. 8(1): p. 64-75.
28. Jiang, W., et al., Nanoparticle-mediated cellular response is size-dependent. Nat Nanotechnol. 2008 Mar;3(3): 145-50. doi: 10.1038/nnano.2008.30. Epub 2008 Mar 2.
29. Rho, K.S., et al., Electrospinning of collagen nanofibers: effects on the behavior of normal human keratinocytes and early-stage wound healing. Biomaterials, 2006. 27(8): p. 1452-61.
30. Boateng, J.S., et al., Wound healing dressings and drug delivery systems: a review. J Pharm Sci, 2008. 97(8): p. 2892-923.
31. Tungprapa, S., I. Jangchud, and P. Supaphol, Release characteristics of four model drugs from drug-loaded electrospun cellulose acetate fiber mats. Polymer, 2007. 48(17): p. 5030-5041.
32. Zhang, X., K. Tang, and X. Zheng, Electrospinning and rheological behavior of poly (vinyl alcohol)/collagen blended solutions. Journal of Wuhan University of Technology-Mater. Sci. Ed., 2015. 30(4): p. 840-846.
33. Koosha, M. and H. Mirzadeh, Electrospinning, mechanical properties, and cell behavior study of chitosan/PVA nanofibers. Journal of Biomedical Materials Research Part A, 2015. 103(9): p. 3081-3093.
34. Chen, X., et al., Synthesis of amphiphilic alginate derivatives and electrospinning blend nanofibers: a novel hydrophobic drug carrier. Polymer Bulletin, 2015. 72(12): p. 3097-3117.
35. Ji, H., et al., Electrospinning and characterization of medium-molecular-weight poly(vinyl alcohol)/high-molecular-weight poly(vinyl alcohol)/montmorillonite nanofibers. Colloid and Polymer Science, 2009. 287(7): p. 751-758.
36. Lee, H.W., et al., Electrospinning and characterisation of poly (vinyl alcohol) blend submicron fibres in aqueous solutions. Polymers & Polymer Composites, 2009. 17(1): p. 47.
37. Zhang, C., et al., Study on morphology of electrospun poly(vinyl alcohol) mats. European Polymer Journal, 2005. 41(3): p. 423-432.
38. Park, J.-C., et al., Electrospun poly(vinyl alcohol) nanofibers: effects of degree of hydrolysis and enhanced water stability. Polym J, 2010. 42(3): p. 273-276.
39. Jannesari, M., et al., Composite poly(vinyl alcohol)/poly(vinyl acetate) electrospun nanofibrous mats as a novel wound dressing matrix for controlled release of drugs. 2011. p. 993-1003.
40. Hong, K.H., et al., Preparation of antimicrobial poly(vinyl alcohol) nanofibers containing silver nanoparticles. Journal of Polymer Science Part B: Polymer Physics, 2006. 44(17): p. 2468-2474.
41. Porel, S., et al., Nanoparticle-Embedded Polymer: In Situ Synthesis, Free-Standing Films with Highly Monodisperse Silver Nanoparticles and Optical Limiting. Chemistry of Materials, 2005. 17(1): p. 9-12.
42. Chun, J.Y., et al., Epidermal cellular response to poly(vinyl alcohol) nanofibers containing silver nanoparticles. Colloids and Surfaces B: Biointerfaces, 2010. 78(2): p. 334-342.
43. Luo, L.-B., et al., Large-Scale Fabrication of Flexible Silver/Cross-Linked Poly(vinyl alcohol) Coaxial Nanocables by a Facile Solution Approach. Journal of the American Chemical Society, 2005. 127(9): p. 2822-2823.
44. De Jaeghere, W., et al., Hot-melt extrusion of polyvinyl alcohol for oral immediate release applications. Int J Pharm, 2015. 492(1-2): p. 1-9.
45. Morita, R., R. Honda, and Y. Takahashi, Development of oral controlled release preparations, a PVA swelling controlled release system (SCRS). I. Design Of SCRS and its release controlling factor. J Control Release, 2000. 63(3): p. 297-304.
46. Cozzolino, C.A., et al., Dye release behavior from polyvinyl alcohol films in a hydro-alcoholic medium: Influence of physicochemical heterogeneity. Colloids and Surfaces A: Physicochemical and Engineering Aspects, 2012. 403: p. 45-53.
47. Murphy, D.J., et al., Physical characterisation and component release of poly(vinyl alcohol)-tetrahydroxyborate hydrogels and their applicability as potential topical drug delivery systems. Int J Pharm, 2012. 423(2): p. 326-34.
48. Pattama, T., R. Uracha, and S. Pitt, Drug-loaded electrospun mats of poly(vinyl alcohol) fibres and their release characteristics of four model drugs. Nanotechnology, 2006. 17(9): p. 2317.

1. A method of forming a polymer matrix, comprising:
admixing a first polyvinyl alcohol (PVA) polymer with a second PVA polymer, wherein the first PVA polymer is hydrolyzed to a first degree of hydrolysis of from 80% to 100% and the second PVA polymer is hydrolyzed to a second degree of hydrolysis of from 75% to 96%, and the first degree of hydrolysis is at least 4% higher than the second degree of hydrolysis, wherein the first and second PVA polymers are present respectively in a blended PVA weight ratio of from 5:95 to 95:5; and,
allowing the admixed PVA polymers to form the polymer matrix, wherein the polymer matrix is at least partially water soluble, and the blended PVA weight ratio is selected to provide a desired degree of water solubility of the matrix.

2. The method of claim 1, wherein the polymer matrix is a hydrogel-forming polymer matrix, forming a hydrogel when appropriately hydrated.

3. The method of claim 1 or 2, wherein the admixing is in the substantial absence of a cross linking agent.

4. The method of any one of claims 1 to 3, wherein the first and second PVA polymers are substantially free of covalent crosslinks therebetween.

5. The method of any one of claims 1 to 4, wherein the admixing comprises electrospinning.

6. The method of any one of claims 1 to 4, wherein the admixing comprises casting and drying.

7. The method of any one of claims 1 to 6, wherein the polymer matrix is substantially free of polyethylene glycol (PEG).

8. The method of any one of claims 1 to 7, wherein polymers in the polymer matrix consist essentially of the first and second PVA polymers.

9. The method of any one of claims 1 to 8, wherein the first PVA polymer and/or the second PVA polymer has a molecular weight of between 9,000 and 150,000.

10. The method of any one of claims 1 to 9, wherein the first degree of hydrolysis is from 90% to 99%, 94% to 99%, or about 99%.

11. The method of any one of claims 1 to 10, wherein the second degree of hydrolysis is less than 99%, 80% to 96%, 88% to 96%, or about 88%.

12. The method of any one of claims 1 to 9, wherein the first degree of hydrolysis is at least 97% and the second degree of hydrolysis is from 90% to 97%; or, the first degree of hydrolysis is 99% and the second degree of hydrolysis is less than 90%; or, the first degree of hydrolysis is 90-97% and the second degree of hydrolysis is less than 90%; or, the first degree of hydrolysis is 99% and the second degree of hydrolysis is 90-97%.

13. The method of any one of claims 1 to 9, wherein the first degree of hydrolysis is from 90% to 99% and the second degree of hydrolysis is below 90%.

14. The method of any one of claims 1 to 13, wherein the first and second PVA polymers are present respectively in the blended PVA weight ratio of from 10:90 to 50:50.

15. The method of any one of claims 1 to 14, wherein the polymer matrix further comprises one or more additional distinct PVA polymers, the additional distinct PVA polymers having a degree of hydrolysis that is different from the first and second degrees of hydrolysis.

16. The method of any one of claims 1 to 15, wherein the polymer matrix is biocompatible.

17. The method of any one of claims 1 to 16, wherein the polymer matrix further comprises a therapeutic agent, a cosmetic agent or a biologically active agent.

18. The method of claim 17, wherein the therapeutic agent is one or more of an antimicrobial agent, an anesthetic agent, an anti-inflammatory agent, an antiproliferative agent or a wound modulating agent.

19. The method of claim 17, wherein the therapeutic agent is a silver salt.

20. The method of claim 19, wherein the silver salt is silver nitrate, silver carbonate, silver sulphate, silver acetate or silver sulphadiazine.

21. The method of any one of claims 17 to 20, wherein the polymer matrix is a controlled release matrix for the therapeutic agent, the cosmetic agent or the biologically active agent.

22. The method of claim 21, wherein the controlled release matrix when applied to a subject releases the therapeutic agent, cosmetic agent or biologically active agent over a slow release period of at least 5, 10 or 15 days.

23. The method of claim 22, wherein the controlled release matrix is topically applied to the subject.

24. The method of any one of claims 1 to 23, wherein the polymer matrix is formed into a skin coating or wound dressing.

25. A polymer matrix, comprising:
a first polyvinyl alcohol (PVA) polymer admixed with a second PVA polymer, wherein the first PVA polymer is hydrolyzed to a first degree of hydrolysis of from 80% to 100% and the second PVA polymer is hydrolyzed to a second degree of hydrolysis of from 75% to 96%, and the first degree of hydrolysis is at least 4% higher than the second degree of hydrolysis, wherein the first and second PVA polymers are present respectively in a blended PVA weight ratio of from 5:95 to 95:5;
wherein the admixed PVA polymers form the polymer matrix, and the polymer matrix is at least partially water soluble, and the blended PVA weight ratio modulates a desired degree of water solubility of the matrix.

26. The polymer matrix of claim 25, wherein the polymer matrix is a hydrogel-forming polymer matrix, forming a hydrogel when appropriately hydrated.

27. The polymer matrix of claim 25 or 26, wherein the polymer matrix substantially lacks a cross linking agent.

28. The polymer matrix of any one of claims 25 to 27, wherein the polymer matrix is substantially free of covalent crosslinks between the first and second PVA polymers.

29. The polymer matrix of any one of claims 25 to 28, wherein the polymer matrix is an electrospun polymer matrix.

30. The polymer matrix of any one of claims 25 to 28, wherein the polymer matrix is a cast and dried polymer matrix.

31. The polymer matrix of any one of claims 25 to 30, wherein the polymer matrix is substantially free of polyethylene glycol (PEG).

32. The polymer matrix of any one of claims 25 to 31, wherein polymers in the polymer matrix consist essentially of the first and second PVA polymers.

33. The polymer matrix of any one of claims 25 to 32, wherein the first PVA polymer and/or the second PVA polymer has a molecular weight of between 9,000 and 150,000.

34. The polymer matrix of any one of claims 25 to 33, wherein the first degree of hydrolysis is from 90% to 99%, 94% to 99%, or about 99%.

35. The polymer matrix of any one of claims 25 to 34, wherein the second degree of hydrolysis is less than 99%, 80% to 96%, 88% to 96%, or about 88%.

36. The polymer matrix of any one of claims 25 to 33, wherein the first degree of hydrolysis is at least 97% and the second degree of hydrolysis is from 90% to 97%; or, the first degree of hydrolysis is 99% and the second degree of hydrolysis is less than 90%; or, the first degree of hydrolysis is 90-97% and the second degree of hydrolysis is less than 90%; or, the first degree of hydrolysis is 99% and the second degree of hydrolysis is 90-97%.

37. The polymer matrix of any one of claims 25 to 33, wherein the first degree of hydrolysis is from 90% to 99% and the second degree of hydrolysis is below 90%.

38. The polymer matrix of any one of claims 25 to 37, wherein the first and second PVA polymers are present respectively in the blended PVA weight ratio of from 10:90 to 50:50.

39. The polymer matrix of any one of claims 25 to 38, wherein the polymer matrix further comprises one or more additional distinct PVA polymers, the additional distinct PVA polymers having a degree of hydrolysis that is different from the first and second degrees of hydrolysis.

40. The polymer matrix of any one of claims 25 to 39, wherein the polymer matrix is biocompatible.

41. The polymer matrix of any one of claims 25 to 40, wherein the polymer matrix further comprises a therapeutic agent, a cosmetic agent or a biologically active agent.

42. The polymer matrix of claim 41, wherein the therapeutic agent is one or more of an antimicrobial agent, an anesthetic agent, an anti-inflammatory agent, an antiproliferative agent or a wound modulating agent.

43. The polymer matrix of claim 41, wherein the therapeutic agent is a silver salt.

44. The polymer matrix of claim 43, wherein the silver salt is silver nitrate, silver carbonate, silver sulphate, silver acetate or silver sulphadiazine.

45. The polymer matrix of any one of claims 41 to 44, wherein the polymer matrix is a controlled release matrix for the therapeutic agent, the cosmetic agent or the biologically active agent.

46. The polymer matrix of claim 45, wherein the controlled release matrix when applied to a subject releases the therapeutic agent, cosmetic agent or biologically active agent over a slow release period of at least 5, 10 or 15 days.

47. The polymer matrix of claim 46, wherein the controlled release matrix is topically applied to the subject.

48. The polymer matrix of any one of claims 25 to 47, wherein the polymer matrix is formed into a skin coating or wound dressing.

49. Use of the polymer matrix of any one of claims 25 to 48, for controlled release of a medicament.

50. The use according to claim 49, wherein the controlled release is topical.

51. A method of treating a subject for a disease or disorder, comprising applying to the subject the polymer matrix of any one of claims 25 to 50.

52. The method of claim 51, wherein the polymer matrix is topically applied to the subject.

53. The method of claim 51 or 52, wherein the polymer matrix comprises a medicament.

54. The method of any one of claims 51 to 53, wherein the subject is a human patient.

55. The method of any one of claims 51 to 54, wherein the disease or disorder is a wound or skin lesion.
